(19) United States
(12) Patent Application Publication
(10) Pub. No.: US 2003/0113292 A1
(43) Pub. Date: Jun. 19, 2003

(54) Methods of treating viral diseases with il-18 and il-18 combinations

(76) Inventors: Klaus M Esser, Collegeville, PA (US); Martin Rosenberg, Collegeville, PA (US); Ruth Tal-Singer, Collegeville, PA (US); Gary Woodnutt, Collegeville, PA (US); Francis V Chisari, La Jolla, CA (US); Susan B Dillon, Alamo, CA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/297,136

(22) PCT Filed: Jun. 1, 2001
(86) PCT No.: PCT/US01/17924

(30) Foreign Application Priority Data
Jun. 2, 2000 (US) 60208869

(51) Int. Cl. A61K 38/21; A61K 39/12; A61K 31/522; A61K 31/7056; A61K 31/513

(52) U.S. Cl. 424/85.1; 424/85.7; 424/186.1; 514/43; 514/263.38; 514/269

(57) ABSTRACT

The present invention relates generally to the use of compositions comprising IL-18, also known as interferon-γ-inducing factor (IGIF), and IL-18 in combination with other agents, for the prevention and/or treatment of viral diseases caused by HIV, HSV, HPV, HAV, HBV, and HCV.

### FIELD OF THE INVENTION

The present invention relates generally to the use of IL-18, also known as interferon-γ-inducing factor (IGIF), and IL-18 in combination with other agents, in the prevention and/or treatment of viral diseases.

### BACKGROUND OF THE INVENTION

IL-18 is a recently discovered novel cytokine. Active IL-18 contains 157 amino acid residues. It has potent biological activities, including induction of interferon-γ-production by T cells and splenocytes, enhancement of the killing activity of NK cells and promotion of the differentiation of naive CD4⁺T cells into Th1 cells. In addition, human IL-18 augments the production of GM-CSF and decreases the production of IL-10. IL-18 has been shown to have greater interferon-γ inducing capabilities than IL-12, and appears to have different receptors and utilize a distinct signal transduction pathway.

CD4⁺T cells are the central regulatory elements of all immune responses. They are divided into two subsets, Th1 and Th2. Each subset is defined by its ability to secrete different cytokines. Interestingly, the most potent inducers for the differentiation are cytokines themselves. The development of Th2 cells from naive precursors is induced by IL-4. Prior to the discovery of IL-18, IL-12 was thought of as the principal Th1 inducing cytokine. IL-18 is also a Th1 inducing cytokine and is more potent than IL-12 in stimulating the production of interferon-γ.

Th1 cells secrete IL-2, interferon-γ, and TNF-β. Interferon-γ, the signature Th1 cytokine, acts directly on macrophages to enhance their microbiocidal and phagocytic activities. As a result, the activated macrophages can efficiently destroy intracellular pathogens and tumor cells. The Th2 cells produce IL-4, IL-5, IL-6, IL-10 and IL-13, which act by helping B cells develop into antibody-producing cells. Taken together, Th1 cells are primarily responsible for cell-mediated immunity, while Th2 cells are responsible for humoral immunity.

IL-18, the encoding nucleotide sequence and certain physicochemical chemical properties of the purified protein is known.

Kabushiki Kaisha Hayashibara Seibutsu Kayaku Kenkyujo's (“Hayashibara”), U.S. Pat. No. 5,912,324, which corresponds to EP 0 692 536 published on Jan. 17, 1996, discloses a mouse protein which induces IFN-gamma production by immunocompetent cells, the protein being further characterized as having certain physicochemical properties and a defined partial amino acid sequence. Also disclosed is a protein having a 157 aa sequence, two fragments thereof, DNA (471 bp) encoding the protein, hybridomas, protein purification methods, and methods for detecting the protein.

Hayashibara's U.S. Pat. No. 6,214,584, which corresponds to EP 0 712 931 published on May 22, 1996, discloses a 157 aa human protein and homologues thereof, DNA encoding the protein, transformants, processes for preparing the protein, monoclonal antibodies against the protein, hybridomas, protein purification methods, methods for detecting the protein, and methods of treatment and/or prevention of malignant tumors, viral diseases, bacterial infectious diseases, and immune diseases.

Incyte Pharmaceuticals, Inc.'s, WO 97/24441, published on Jul. 10, 1997, discloses a 193 aa protein corresponding to IL-18 precursor and encoding DNA.

Viral diseases, such as HIV, HSV, HPV, HAV, HVB and HCV are presently treated and/or prevented with, for example, antiviral agents, immunotherapy and vaccines. Current treatments, however, are not always effective. There is a need, therefor, for a more effective treatment for such viral diseases.

### SUMMARY OF THE INVENTION

In one aspect, the present invention provides a method of treating and/or preventing viral disease, such as HIV, HSV, HPV, HAV, HBV and HCV, comprising administering a viral disease inhibiting amount of a polypeptide having at least 70% identity of the amino acid sequence of SEQ ID NO:1 or SEQ ID NO:2 over the entire length of the sequences alone or in combination with antiviral agents, such as but not limited to foscarnet, acyclovir (ACV), ACV-phosphonate, brivudine (bromovinyldeoxyuridine, BVDU), cidofovir (HPMPC, GS504), cyclic HPMPC, famciclovir, ganciclovir (GCV), GCV-phosphonate, lobucavir (bishydroxymethylcyclobutylguanine, BHCG), penciclovir, ribavirin, adefovir, lamivudine (3TC), abacavir, stavudine, zidovudine, tenovir, other cytokines, such as IL-2, IL-12, IFN or immunomodulators such as but not limited to ribavirin, thymosin alpha, corticosteroids, thalidomide, imiquimod, as well as with vaccines such as but not limited to Havrix®, Engerix®.

In a further aspect, the present invention provides a method of preventing and/or treating a viral disease such as HIV, HSV, HPV, HAV, HBV and HCV in a mammal comprising the administration of a viral disease inhibiting amount of a composition comprising IL-18, alone or in combination with antiviral agents, such as but not limited to foscarnet, acyclovir (ACV), ACV-phosphonate, brivudine (bromovinyldeoxyuridine, BVDU), cidofovir (HPMPC, GS504), cyclic HPMPC, famciclovir, ganciclovir (GCV), GCV-phosphonate, lobucavir (bishydroxymethylcyclobutylguanine, BHCG), penciclovir, ribavirin, adefovir, lamivudine (3TC), abacavir, stavudine, zidovudine, tenovir, other cytokines, such as IL-2, IL-12, IFN or immunomodulators such as but not limited to ribavirin, thymosin alpha, corticosteroids, thalidomide, imiquimod, as well as with vaccines such as but not limited to Havrix®, Engerix®. The composition may also include a pharmaceutically acceptable carrier.

### BRIEF DESCRIPTION OF THE DRAWINGS

FIG. 1 shows the amino acid sequence of human IL-18 (Sequence ID NO:1).

FIG. 2 shows the amino acid sequence of murine IL-18 (Sequence ID NO:2).

FIG. 3 shows graphs demonstrating the induction of IFN-γ protein in mice treated with varying amounts of murine IL-18 administered intraperitoneally in buffered saline.

FIG. 4 shows graphs demonstrating the induction of IFN-γ mRNA in mice treated with varying amounts of murine IL-18 administered intraperitoneally in buffered saline.

FIG. 5 shows a graph demonstrating improved survival of mice challenged with a lethal dose of HSV-1 (SC-16) following intraperitoneal administration of murine IL-18 at −2 h, 1 day and 2 days compared to controls.

FIG. 6 is a graph showing that the administration of IL-18 lead to improvement in influenza-induced weight loss

FIG. 7 is a graph showing that the administration of IL-18 lead to improvement in pulmonary functions measured using pulse oximetry.

FIG. 8 shows the effect of IL-18 on HBV replication

FIGS. 9(a)-9(d) are graphs showing that IL-18 induced IL-8 14-fold (FIG. 9(a)), Neopterin 7-fold (FIG. 9(b)), GM-CSF 100-fold (FIG. 9(c)), and IFN-gamma 8-fold (FIG. 9(d)).

FIGS. 10(a)-10(c) are graphs showing that IL-18 induced the production of IFN-gamma, (FIG. 10(a)), Neopterin (FIG. 10(b)), and IL-8 (FIG. 10(c)). In this study, treatment with IL-2 alone was used as control.

FIG. 11 shows the effect of IL-12 and IL-18 on HBV replication

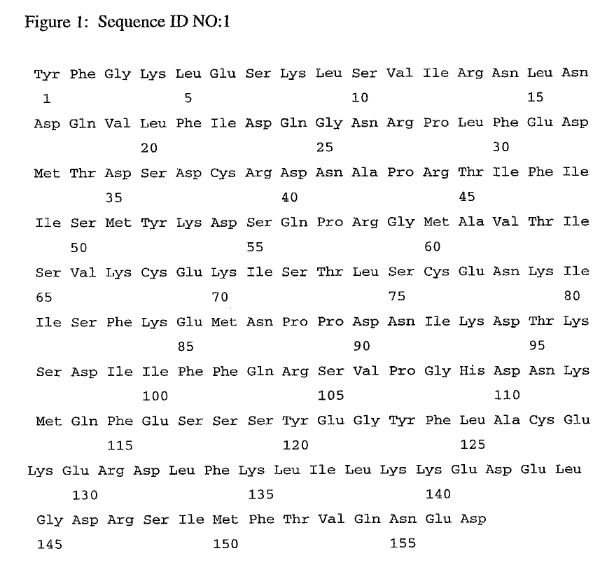

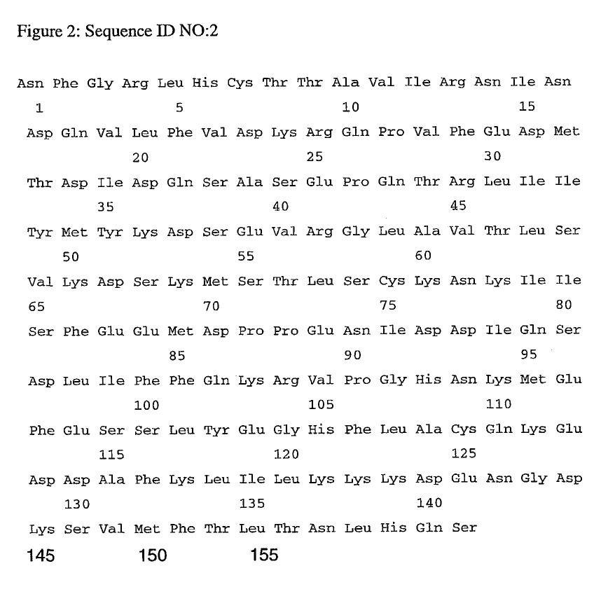

### DETAILED DESCRIPTION OF THE INVENTION

The present invention relates generally to methods of treating and/or preventing viral diseases such as HIV, HSV, HPV, HAV, HBV and HCV, comprising administering a viral disease inhibiting amount of IL-18 and compositions comprising IL-18.

The following definitions are provided to facilitate understanding of certain terms and abbreviations used frequently in this application.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, PROTEINS—STRUCTURE AND MOLECULAR PROPERTIES, 2nd Ed., T. E. Creighton, W. H. Freeman and Company, New York, 1993; Wold, F., Post-translational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter et al., “Analysis for protein modifications and nonprotein cofactors”, Meth Enzymol (1990) 182:626-646 and Rattan et al., “Protein Synthesis: Post-translational Modifications and Aging”, Ann NY Acad Sci (1992) 663:48-62).

“Variant” refers to a polynucleotide or polypeptide that differs from a reference polynucleotide or polypeptide, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

Preferred parameters for polypeptide sequence comparison include the following:

1) Algorithm: Needleman and Wunsch, J. Mol Biol. 48: 443-453 (1970)

Comparison matrix: BLOSSUM62 from Hentikoff and Hentikoff, Proc. Natl. Acad. Sci. USA. 89:10915-10919 (1992)

Gap Penalty: 12

Gap Length Penalty: 4

A program useful with these parameters is publicly available as the “gap” program from Genetics Computer Group, Madison Wis. The aforementioned parameters are the default parameters for peptide comparisons (along with no penalty for end gaps).

A polypeptide sequence of the present invention may be identical to the reference sequence of SEQ ID NO:1 or SEQ ID NO:2, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in SEQ ID NO:1 or SEQ ID NO:2 by the numerical percent of the respective percent identity (divided by 100) and then subtracting that product from said total number of amino acids in SEQ ID NO:1 or SEQ ID NO:2, respectively, or:

nₐ≦xₐ−(xₐ·y),

wherein nₐ is the number of amino acid alterations, xₐ is the total number of amino acids in SEQ ID NO:1 or SEQ ID NO:2, and y is, for instance 0.70 for 70%, 0.80 for 80%, 0.85 for 85% etc., and wherein any non-integer product of xₐ and y is rounded down to the nearest integer prior to subtracting it from xₐ.

“Fusion protein” refers to a protein encoded by two, often unrelated, fused genes or fragments thereof. In one example, EP-A-0 464 discloses fusion proteins comprising various portions of constant region of immunoglobulin molecules together with another human protein or part thereof. In many cases, employing an immunoglobulin Fc region as a part of a fusion protein is advantageous for use in therapy and diagnosis resulting in, for example, improved pharmacokinetic properties [see, e.g., EP-A 0232 262]. On the other hand, for some uses it would be desirable to be able to delete the Fc part after the fusion protein has been expressed, detected and purified.

IL-18 Polypeptide

The IL-18 polypeptide is disclosed in EP 0692536A2, EP 0712931A2, EP0767178A1, and WO 97/2441. The polypeptides include isolated polypeptides comprising an amino acid sequence which has at least 70% identity, preferably at least 80% identity, more preferably at least 90% identity, yet more preferably at least 95% identity, most preferably at least 97-99% identity, to that of SEQ ID NO:1 (human IL-18) and SEQ ID NO:2 (murine IL-18) over the entire length of SEQ ID NO:1 and SEQ ID NO:2, respectively. Such polypeptides include those comprising the amino acid of SEQ ID NO:1 and SEQ ID NO:2, respectively.

Polypeptides of the present invention are interferon-γ-inducing polypeptides. They play a primary role in the induction of cell-mediate immunity, including induction of interferon-γ production by T cells and spleenocytes enhancement of the killing activity of NK cells and promotion of the differentiation of naive CD4+ T cells into Th1 cells. These properties are hereinafter referred to as “IL-18 activity” or “IL-18 polypeptide activity” or “biological activity of IL-18”. Also included amongst these activities are antigenic and immunogenic activities of said IL-18 polypeptides, in particular the antigenic and immunogenic activities of the polypeptides of SEQ ID NO:1 and SEQ ID NO:2. Preferably, a polypeptide of the present invention exhibits at least one biological activity of IL-18.

The polypeptides of the present invention may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

The present invention also includes variants of the aforementioned polypeptides, that is polypeptides that vary from the referents by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, 1-3, 1-2 or 1 amino acids are substituted, deleted, or added in any combination.

Polypeptides of the present invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

Recombinant polypeptides of the present invention may be prepared by processes well known in the art from genetically engineered host cells comprising expression systems. Accordingly, in a further aspect, the present invention relates to expression systems which comprises a polynucleotide or polynucleotides encoding the polypeptides of the present invention, to host cells which are genetically engineered with such expression systems and to the production of polypeptides of the invention by recombinant techniques. Cell-free translation systems can also be employed to produce such proteins using RNAs derived from the DNA constructs of the present invention.

Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector which is able to maintain, propagate or express a polynucleotide to produce a polypeptide in a host may be used. The appropriate nucleotide sequence may be inserted into an expression system by any of a variety of well-known and routine techniques, such as, for example, those set forth in Sambrook et al., Molecular Cloning: A Laboratory Manual, 2nd Ed., Cold Spring Harbor Laboratory Press, Cold Spring Harbor, N.Y. (1989). Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals.

Polypeptides of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, high performance liquid chromatography, hydroxylapatite chromatography and lectin chromatography. Most preferably, affinity chromatography is employed for purification. Well-known techniques for refolding proteins may be employed to regenerate active conformation when the polypeptide is denatured during isolation and or purification.

The therapeutic potentials for IL-18 in the prevention/treatment of certain viral diseases have been evaluated in animal models, and protective effects have been demonstrated. Administration of IL-18 to normal, nude or SCID mice improved survival in HSV-1 infections; protection was mediated at least in part via IFNγ (Fujioka, et al 1999 J. Virology 73:2401). In addition, recent data indicate that IFNγ is important for rapid suppression of HSV following reactivation from latency (Cantin, et al 1999 J. Virology 73:5196; Cantin, et al 1999 J. Virology 73:3418) suggesting a potential for IL-18 therapy for suppression of recurrent disease due to reactivation. Vaccinia virus-induced pock formation was reduced in response to IL-18 treatment, a result consistent with the enhanced replication of vaccinia virus in IFNγ receptor knock-out mice. In both herpes and vaccinia viral infection models, IL-18 was administered either prophylactically, and/or early after infection.

Polypeptides of the present invention can be used alone or can be combined with antiviral agents, other cytokines, IFN, antibiotics or antiviral vaccines to treat and or prevent various viral diseases.

### HIV

In the instance of HIV infection, polypeptides of the present invention can be used alone or can be combined with protease inhibitors (PIs), nucleoside analog reverse transcriptase inhibitors (NRTIs), non-nucleoside reverse transcriptase inhibitors (NNRTIs), HIV receptor or co-receptor inhibitors, fusion inhibitors, antisense oligonucleotide inhibitors, glucosidase inhibitors, other cytokines, IFN, antibiotics or immunomodulatory agents. Examples of PIs include but are not limited to amprenavir, crixivan, DMP-323, DMP-450, indinavir, KNI-272, lasinavir, lopinavir, viracept, PD178390, ritonavir, RPI 312, saquinavir, SC-52151, SDZ PRI 053, tipranavir, U-103017, and A-77003. Examples of NRTIs include but are not limited to abacavir, adefovir, alovudine, AZdU, CS-92, DAPD, didanosine, dOTC, coviracil, lamivudine, lobucavir, iodenosine, stavudine, tenofovir, zalcitabine, and zidovudine. Examples of NNRTIs include but are not limited to atevirdine mesylate, calanolide A, capravirine, delavirdine, efavirenz, emivirine, GW420 867X, HBY 097, loviride, nevirapine, PETT-5, tivirapine, and trovirdine. Examples of receptor, co-receptor and fusion inhibitors include but are not limited to AMD 3100, TAK 779, T-20 and T-1249. Examples of these and further antiviral agents of various mechanisms of action against HIV can be found periodically summarized in International Antiviral News, for example in volume 8, number 1, January 2000. Examples of cytokines and immunomodulatory agents include but are not limited to IL-2, steroids, and thalidomide. The current invention can also be administered as monotherapy to infected individuals in order to enhance the natural immune response, to achieve either control or clearance of the infection.

### HSV

Herpes simplex virus is a member of the Herpesviridae family that typically infects mucosal surfaces or the skin. Latency is established in neurons of the sensory and autonomic ganglia. Under certain stimuli such as stress, fever, UV radiation or immunosuppression, the virus can reactivate and appear at the original site of infection or at any site innervated by the ganglion. Antiviral agents are currently available and highly effective at inhibiting alphaherpesvirus replication. However, although they provide a modest reduction in healing time, there is limited beneficial effect on establishment of viral latency. Recent data indicate that IFN-γ is important for rapid suppression of HSV following reactivation from latency (Cantin et al, Journal of Virology 73:3418-3423, 73:5196-5200) suggesting a potential for IL-18 therapy for suppression of recurrent disease due to reactivation.

Herpes simplex encephalitis (HSE) is a severe sporadic disease that accounts for 10-20% of viral encephalitis cases. It is the most severe form of herpes simplex virus (HSV) infection causing focal, necrotizing lesions that in many cases result in severe neurological sequelae (Whitley and Roizman 1998, Clin. Inf. Dis. 26:541-547). Both HSV-1 and 2 have been associated with infections of the central nervous system (CNS). Antiviral treatment may reduce HSE-associated mortality to approximately 30%, but may still leave survivors with severe neurological impairment (Skoldenberg, 1996 Sc. J. Inf. Dis. 100:8-13; Kimberlin et al. 1998 J. Neurovirology 4:474-485).

Polypeptides of the present invention can be used alone or in combination with antivirals such as viral polymerase inhibitors (exemplified by acyclovir, valacylcovir, penciclovir, famciclovir, ganciclovir, vangancilcovir, foscarnet, codofovir, and other nucleosides and nucleotides). The polypeptides can also be used in combination with cytokines such as IFN, IL-2, IL-12 and others, as well as with other immunomodulators. The current invention can also be administered as monotherapy to infected individuals in order to enhance the natural immune response, to achieve either control or clearance of the infection.

### HPV

There is an unmet need for preventing and/or treating HPV infections. To date, over 100 HPV types have been identified. Infection can be asymptomatic, can produce warts or may result in various benign or malignant genital neoplasias including cervical carcinoma (reviewed by Koutsky, Am. J. Med. 102: 3-8, 1997). Although accurate figures are not available, it has been estimated that visible genital warts are present in 1% of the sexually active adults in the US, and that at least 15% have molecular evidence of HPV infection. The result is approximately 65,000 cases of cervical and genital carcinoma per year. Currently there are no antiviral drugs available and HPV disease is treated by chemical or physical ablation, cytotoxic agents or immunotherapy (Miller, R. L. et al. Int J Immunopharmacology 21: 1-14, 1999). Although most of the current therapies are eventually able to clear warts in a majority of patients, they do not impact virus transmission or disease progression (Beutner and Ferenczy, Am. J. Med. 102: 28-37, 1997). Women with abnormal pap smears and a high risk HPV serotype could potentially be identified for treatment with the prospect of inducing a lasting immunity and preventing development of cervical carcinoma.

Polypeptides of the current invention can be used in combination with antivirals such as cidofovir (HPMPC, GS504), BVDU, BVRU, and other nucleosides and nucleotides. The current invention can also be used in combination with other immunomodulators such as interferon or interferon inducers (imiquimod, Aldara; IL-12). The present invention can also be used in combination with recombinant vaccines (either preventative or therapeutic) currently in development. The current invention can also be administered as monotherapy to infected individuals in order to enhance the natural immune response, to achieve either control or clearance of the infection.

Recent data suggest a potentially protective role for IL-18 in herpesvirus-related oncogenesis. IL-18, as well as interferon gamma, are upregulated in EBV acutely-infected cells, and downregulated in post-transplant lymphoproliferative disease induced by EBV ( Setsuda et al. 1999. American Journal of Pathology 155:257-265). These data suggest that these mediators are involved in host defense against the oncogenic properties of EBV. The present invention can be used in combination with agents such as those listed for HSV. The current invention can also be administered as monotherapy to infected individuals in order to enhance the natural immune response, to achieve either control or clearance of the infection.

### HAV

The hepatitis A Virus (HAV) belongs to the picornavirus family. Hepatitis A is highly contagious from person-to person via the fecal-oral route through contaminated food, food handlers, contaminated water, ingestion of shellfish from contaminated water, and by other direct human-to-human contact. HAV replicates in the liver and is excreted in the bile. Infection is acute and generally symptomatic, with symptoms ranging from mild and transient to severe and prolonged, and may include fever, vomiting, diarrhea, jaundice and hepatomegaly. Treatment is generally supportive, with liver transplantation performed rarely in especially severe cases. Prevention is via pre-exposure active immunization with inactivated virus (Havrix) or post exposure passive immunization with pooled immunoglobulin.

Polypeptides of the current invention can be used in combination with either the current vaccines to enhance immunity or to achieve a therapeutic effect (once the patient is already infected). The current invention can also be administered as monotherapy to infected individuals in order to enhance the natural immune response, to achieve either control or clearance of the infection.

### HBV

Hepatitis B virus (HBV) is a member of the Hepatdnaviridae family of DNA viruses. HBV is transmitted from person-to-person via blood or body fluids via similar routes as the transmission of HIV. HBV replicates primarily in the liver, with virus shed into the blood stream and subsequently found in body secretions including semen and saliva. There is an incubation period of 60-180 days between exposure and clinical symptoms, with the latter ranging from asymptomatic infection to cholestatic hepatitis with jaundice, and occasionally liver failure. After the acute course, the majority of patients clear the virus and become immune. Some patients develop chronic infection which can lead to chronic liver disease, fibrosis and hepatocellular carcinoma.

Multiple agents are available form the treatment of HBV, although most are only effective in a fraction of chronic HBV infections. Currently available approved and experimental agents include antivirals (lamivudine[3TC], famciclovir, lobucavir, Adefovir, and a number of other nucleoside and nucleotide agents); immunomodulators (interferon alpha, beta, gamma, corticosteroids, Levamisole, Thymosin alpha, IL-2, ribavirin) and therapeutic vaccines or hyperimmune globulin.

Polypeptides of the current invention can be used in combination with any of these current therapies or other similar agents. The current invention can also be administered as monotherapy to infected individuals in order to enhance the natural immune response, to achieve either control or clearance of the infection.

### HCV

Hepatitis C virus (HCV) is a single stranded RNA virus member of the Hepacivirus or Flavivirus families. HCV is transmitted from person-to-person in much the same way as HIV and HBV, with virus present in blood and body secretions. HCV replicates primarily in the liver, although virus can be found in other cell types such as lymphocytes and dendritic cells. Acute infection is often asymptomatic or characterized by a mild course often confused with more common viral infections. In rare cases, acute infection can lead to fulminant hepatitis and death. Most infections result in a chronic, often asymptomatic infection which may continue for decades with only occasional rises in liver enzymes or mild cirrhosis. Some fraction of cases go on to more severe liver disease to include liver failure and hepatocellular carcinoma.

Treatment of HCV is generally via interferon-alpha, consensus interferon, or interferon-alpha in combination with ribavirin. True antiviral compounds are now just in early clinical trials, and include inhibitors of the viral polymerase, antisense polynucleotides, or ribozymes.

The present invention can be used in combination with any of these current therapies or with other similar agents. The current invention can also be administered as monotherapy to infected individuals in order to enhance the natural immune response, to achieve either control or clearance of the infection.

It is believed that the administration of IL-18 during chronic HCV infection is expected to reduce virus levels through induction of non-cytolytic antiviral cytokines such as IFNγ or TNFα or through enhancement of T-cell responses to viral antigens leading to enhanced and sustained protective immunity.

The present invention also provides for pharmaceutical compositions comprising a therapeutically effective amount of IL-18, optionally in combination with another agent, as described above. Pharmaceutically acceptable carriers or excipients may also be employed. The pharmaceutical carrier employed may be, for example, either a solid or a liquid. Exemplary of solid carriers include, but are not limited to lactose, terra alba, sucrose, talc, gelatin, agar, pectin, acacia, magnesium stearate, stearic acid and the like. Exemplary of liquid carriers include, but are not limited to, saline, buffered saline, dextrose, water, glycerol, ethanol syrup, peanut oil olive oil, and combinations thereof. Similarly, the carrier or diluent may include time delay material well known in the art, such as glyceryl monostearate or glyceryl distearate alone or with a wax ethylcellulose, hydroxypropylmethylcellulose, methylmethacrylate and the like.

The invention further relates to pharmaceutical packs and kits comprising one or more containers filled with one or more of the ingredients of the aforementioned compositions of the invention. The polypeptides may be employed alone or in conjunction with other compounds, such as therapeutic compounds.

The composition will be adapted to the route of administration, for instance by a systemic or an oral route. Preferred forms of systemic administration include injection, typically by intravenous injection. Other injection routes, such as subcutaneous, intramuscular, or intraperitoneal, can be used. In addition, if the present invention can be formulated in an enteric or an encapsulated formulation, oral administration may be possible. Alternative means for systemic administration include transmucosal and transdermal administration using penetrants such as bile salts or fusidic acids or other detergents. Administration of these combinations may also be topical and/or localized, in the form of salves, pastes, gels, and the like.

The dosage range of IL-18 required depends on the choice of adjuvant, if any, the route of administration, the nature of the formulation, the nature of the subject's condition, and the judgment of the attending practitioner. Suitable dosages of the composition, however, for IL-18 are in the range of 1 nanogram/kilogram to 1 milligram/kilogram of subject. Wide variations in the needed dosage, however, are to be expected in view of the variety of compounds available and the differing efficiencies of various routes of administration. For example, transdermal administration would be expected to require higher dosages than administration by intravenous injection. Variations in these dosage levels can be adjusted using standard empirical routines for optimization, as is well understood in the art.

The schedule for the administration of the composition depends on the dosage, on the choice of adjuvant, the route of administration, the nature of the formulation, the nature of the subject's condition, and the judgment of the attending practitioner. Suitable schedules for administration, are daily, weekly, or monthly. Wide variations in the schedules for the administration of the composition, however, are to be expected in view of the variety of other agents available and the differing efficiencies of various routes of administration. For example, transdermal administration would be expected to require higher dosages than administration by intravenous injection. Variations in these schedules for the administration of the composition can be adjusted using standard empirical routines for optimization, as is well understood in the art.

All publications, including but not limited to patents and patent applications, cited in this specification are herein incorporated by reference as if each individual publication were specifically and individually indicated to be incorporated by reference herein as though fully set forth.

It is believed that one skilled in the art can, using the preceding description, utilize the present invention to its fullest extent. Therefore the Examples herein are to be construed as merely illustrative and not a limitation of the scope of the present invention in any way.

### EXAMPLES

### Example 1

### Treatment of Mice with Murine IL-18 Induces IFN γ and GM-CSF

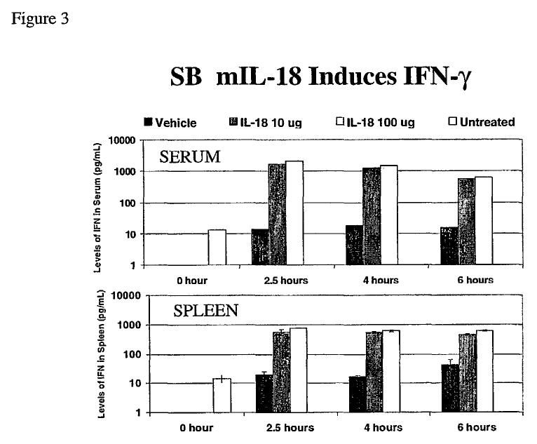

The activity of murine IL-18 was evaluated by profiling the kinetics of cytokine message and protein induction in uninfected mice. Female Balb/C mice were treated intraperitoneally (IP) with 10 or 100 ug murine IL-18 and samples were collected at 0, 2.5, 4, or 6 hours post-treatment. Pooled sera (n=3) and individual spleen homogenates were evaluated by ELISA for TNF-α, IFN-γ, and GM-CSF. IL-18 induced IFN levels in sera and spleens (2 ng/ml) by 2.5 hours post-treatment, consistent with activity. Minimal induction of TNF-α (100 pg/ml) and no induction of GM-CSF were detected. In contrast, mRNA induction of GM-CSF, IFN-gamma, and TNF was observed by quantitative real-time PCR. IFN γ mRNA was induced up to 20 fold over vehicle at 2.5-6 hours post injection (FIG. 3) and GM-CSF was about 6-10 fold induced (not shown) relative to vehicle control values. Thus, the activity of murine IL-18 at inducing cytokines was validated.

FIG. 3 demonstrates induction of IFN-γ protein in mice treated with varying amounts of murine IL-18 administered IP in buffered saline. Protein levels were detected in sera and spleen homogenates using ELISA kits as per manufacturer's instructions (R&D Systems)

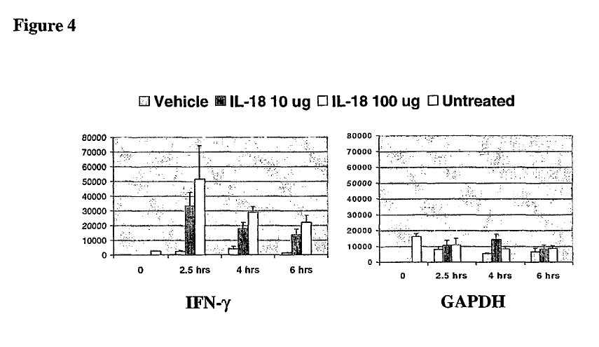

FIG. 4 demonstrates induction of IFN-γ mRNA in mice treated with varying amounts of murine IL-18 administered IP in buffered saline. Total RNA was harvested from spleens and cDNA (prepared using Superscript, Life Technologies) was analyzed in individual samples using real-time PCR for housekeeping gene GAPDH and IFN-65 (method described in R. J. Cohrs et al. J. Virology 2000; 24:11464-11471).

### Example 2

### Murine IL-18 Protects Mice from Lethal HSV-1 Challenge

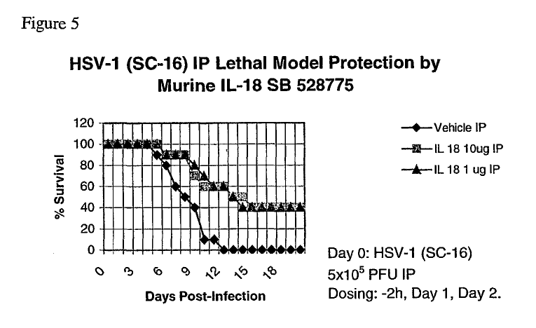

Several studies evaluated the effect of murine IL-18 in a lethal systemic HSV infection model. Murine IL-18 was administered IP at −2 hours, 24 hours, and 48 hours following IP infection with HSV-1 (SC-16). IL-18 treatment at 10-ug/mouse lead to 40% survival in all studies, relative to no survival of vehicle treated animals (FIG. 5). In all studies IL-18 treatment lead to a delay in time to death. In a single study, two daily doses of IL-18 at 100 ug/mouse lead to 70% survival (not shown).

FIG. 5 demonstrates improved survival of mice challenged with a lethal dose of HSV-1 (SC-16) following IP administration of murine IL-18 at −2 h, 1 day and 2 days compared to controls.

### Example 3

### IL-18 Improves Influenza-induced Pathogenesis

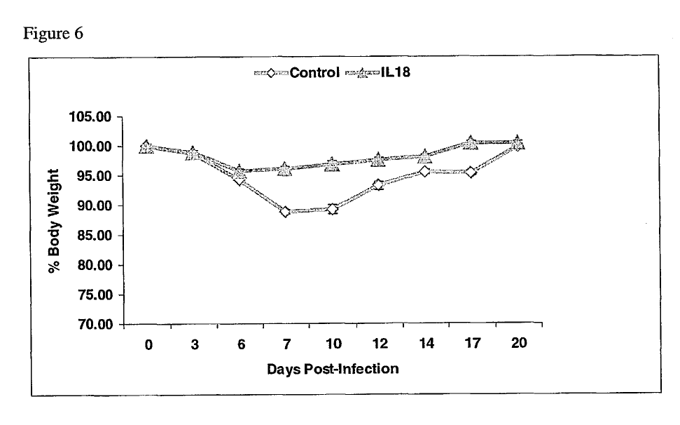

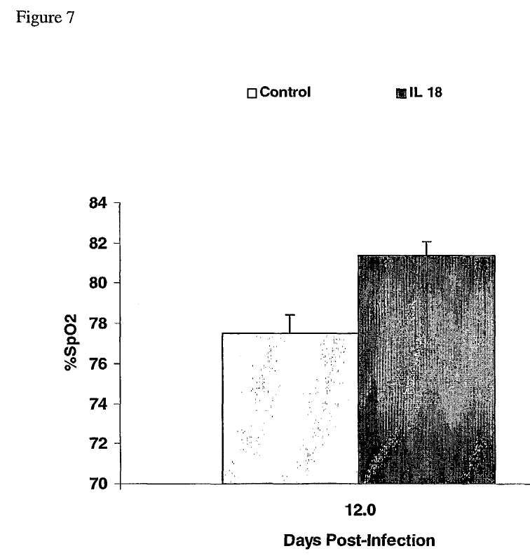

Treatment with IL-18 has a beneficial effect on clinical disease in a murine influenza pneumonia model. Balb/C mice were inoculated intranasally with a sublethal challenge of mouse-adapted Influenza A/PR/8/34. Administration of IL-18 as described in Example 2 lead to improvement in influenza-induced weight loss (FIG. 6), as well as in pulmonary functions measured using pulse oximetry (FIG. 7) and whole body plethysmography (Buxco Electronics, not shown).

### Example 4

### IL-18 Administration Reduces HBV Virus Replication

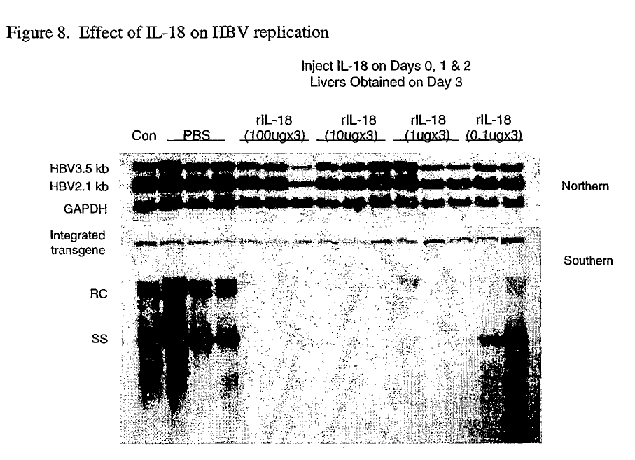

IL-18 Treatment of Hepatitis B Virus (HBV) transgenic mice results in a dose-dependent reduction in virus replication as demonstrated by reduction in the levels of viral DNA (FIG. 8). HBV transgenic mice (Guidotti et. al, 1995. J. Virol 69:6158-6169) were treated with three daily subcutaneous injections (Days 0, 1 and 2) of 4 different doses (100, 10, 1 and 0.1 microgram) of recombinant murine IL-18, with analysis of the liver for HBV DNA on Day 3 by Southern blot. All dose levels of IL-18 had an effect, with some reduction seen at the lowest dose (0.1 micrograms), and greater reductions at higher doses.

### Example 5

### IL-18 Acts Synergistically in Combination with IL-2 for Inducing IFN Production in Chimpanzee and Human Peripheral Blood Mononuclear Cells (PBMCs)

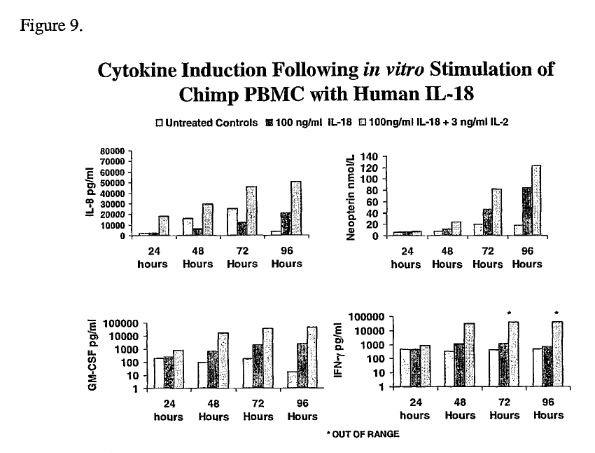

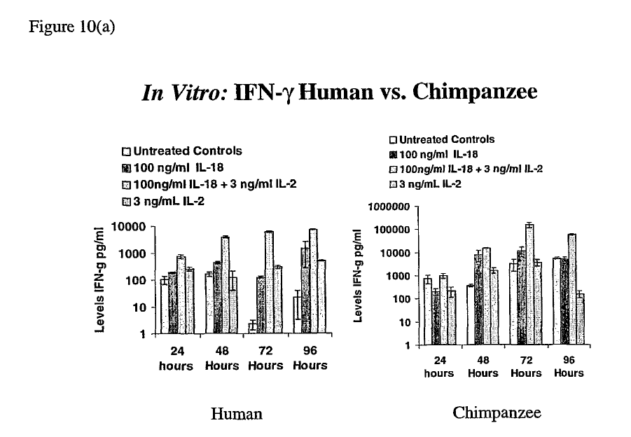

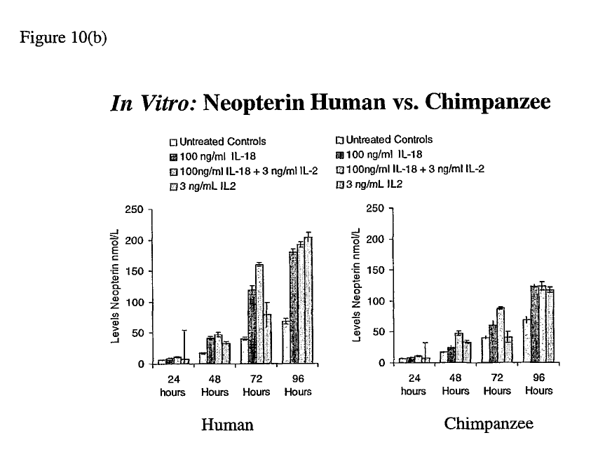

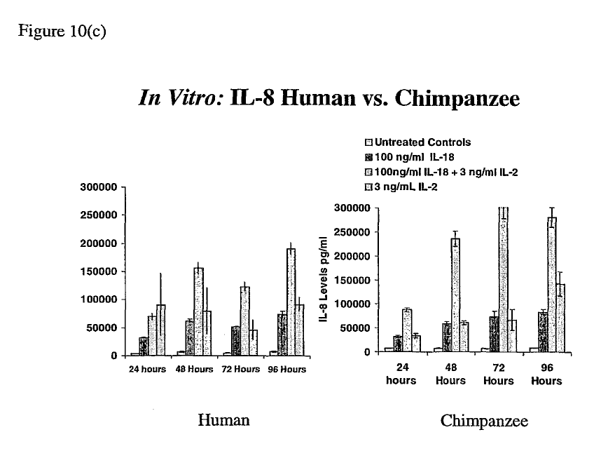

Chimpanzee or human PBMCs were isolated and treated with either control media, human IL-18 at 100 ng/ml, or IL-18 (100 ng/ml) in combination with IL-2 at 3 ng/ml. Supernatants were frozen following incubation for 24, 48, 72, or 96 hours. Mediator levels were analyzed by ELISA. Data shown in FIGS. 9 and 10, demonstrate that human IL-18 is active at inducing cytokine expression in chimp PBMCs with similar kinetics to its effect on human PBMCs. Moreover there is synergy between IL-2 and IL-18 at inducing GM-CSF, IL-8, Neopterin, and IFN-gamma (FIGS. 9 and 10). These observations were confirmed in multiple chimp and human donors.

FIG. 9 demonstrates that human IL-18 induced IL-8 (14-fold), Neopterin (7-fold), GM-CSF (100-fold), and IFN-gamma (8-fold). The highest protein levels were detected in samples obtained 96 hours post-treatment, with the exception of IFN-gamma that reached a maximum level at 24 hours. Combination treatment with human IL-18 and IL-2 lead to significantly higher induction.

FIG. 10 demonstrates the reproducibility of the findings in different animals. In this study, treatment with IL-2 alone was used as control. IFN-gamma is shown in FIG. 10(a); Neopterin in FIG. 10(b); and IL-8 is shown in FIG. 10(c).

### Example 6

### IL-18 Inhibition of HBV Replication is Additive or Synergistic with IL-12

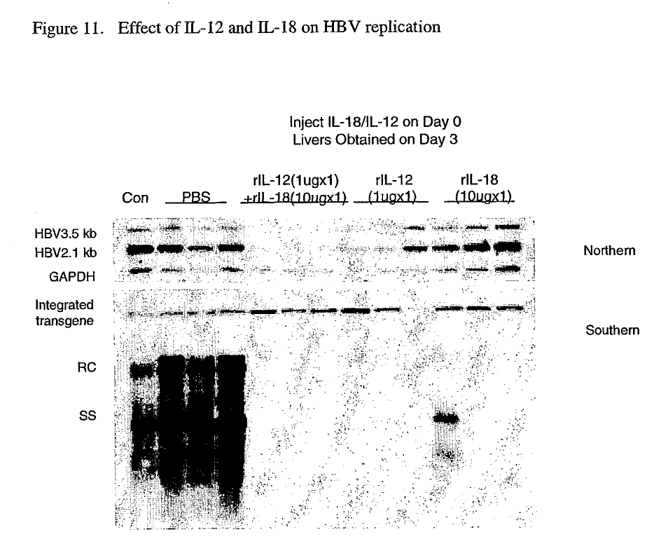

In similar studies to those described in Example 4 and FIG. 8, IL-18 treatment of Hepatitis B Virus (HBV) transgenic mice in combination with IL-12 has greater effects in reducing viral replication as well as transcription of viral DNA, than either cytokine alone. This additive or synergistic effect was seen after a single subcutaneous treatment of HBV transgenic mice on Day 0 with IL-18 (10 microgram) together with IL-12 (1 microgram). When livers of these mice were examined on Day 3, HBV replication was markedly reduced as evident from detection of viral; DNA by Southern blot (FIG. 11). Also, the combination of IL-18 with IL-12 not only reduced HBV DNA production but also dramatically reduced production of viral RNA as evident from Northern blots of these same liver samples (FIG. 11)

What is claimed is:

1. A method of treating a disease caused by Influenza Virus, HIV, HSV, HPV, HAV, HBV or HCV in a mammal comprising the administration of a therapeutically effective amount of a composition comprising a polypeptide having at least 90% identity of the amino acid sequence of SEQ ID NO:1 over the entire length of SEQ ID NO:1.

2. A method of treating a disease caused by Influenza Virus, HIV, HSV, HPV, HAV, HBV or HCV in a mammal comprising the administration of a therapeutically effective amount of a composition comprising a polypeptide having at least 90% identity of the amino acid sequence of SEQ ID NO:2 over the entire length of SEQ ID NO:2.

3. A method of preventing a disease caused by Influenza Virus, HIV, HSV, HPV, HAV, HBV or HCV in a mammal comprising the administration of a therapeutically effective amount of a composition comprising the polypeptide of claim 1.

4. A method of preventing a disease caused by Influenza Virus, HIV, HSV, HPV, HAV, HBV or HCV in a mammal comprising the administration of a therapeutically effective amount of a composition comprising the polypeptide of claim 2.

5. A method of treating a disease caused by a virus comprising the administration of a therapeutically effective amount of a composition comprising the polypeptide of claim 1 or 2 and an antiviral agent.

6. A method of treating a disease caused by a virus comprising the administration of a therapeutically effective amount of a composition comprising the polypeptide of claim 1 or 2 and an immunomodulatory cytokine.

7. A method of treating a disease caused by a virus comprising the administration of a therapeutically effective amount of a composition comprising the polypeptide of claim 1 or 2 and an agent selected from the group consisting of:
ribavirin, interferon α or β, IL-2, IL-12, GM, CSF, TNF, lamivudine, rebetron (ribavirin & interferon α), cidofovir, acylovir, valacylovir, penciclovir, famciclovir, ganciclovir, or valganeciclovir.

8. A method of treating a disease caused by a virus comprising the administration of a therapeutically effective amount of a composition comprising the polypeptide of claim 1 or 2 and an immunogen derived from a viral protein or nucleotide sequence.

9. A method of treating a disease caused by a virus comprising the administration of a therapeutically effective amount of a composition comprising the polypeptide of claim 1 or 2 and a viral vaccine.

10. A method of treating a disease caused by a virus comprising the administration of a therapeutically effective amount of a composition comprising the polypeptide of claim 1 or 2 and a vaccine selected from Havrix, Engerix B and Recombivax.


SEQUENCE LISTING (2 sequences)

SEQ ID NO: 1 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 157 aa, Mus musculus
NFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS